(19) United States
(12) Patent Application Publication
(10) Pub. No.: US 2016/0038127 A1
(43) Pub. Date: Feb. 11, 2016

(54) Biopsy Probe and Use Thereof

(71) Applicant: R. HASHIMSHONY ENGINEERING LTD., Pardes Hana (IL)

(72) Inventor: Rami Hashimshony, Pardes Hana (IL)

(21) Appl. No.: 14/774,565

(22) PCT Filed: Mar. 13, 2014
(86) PCT No.: PCT/IL2014/050267

§ 371 Date: Sep. 10, 2015

Related U.S. Application Data
(60) Provisional application No. 61/786,685, filed on Mar. 15, 2013.

(51) Int. Cl. A61B 10/02; A61B 10/00

(52) U.S. Cl. CPC A61B 10/0275; A61B 10/0096; A61B 2010/0225; A61B 2017/00867

(57) ABSTRACT

There is provided herein a biopsy probe, comprising an outer elongated hollow body having a distal end configured to penetrate a sampling region, a proximal end and an opening located between said distal end and said proximal end, and an inner needle for obtaining one or more tissue samples, wherein at least a part thereof is located is an inner part of said outer elongated hollow body, wherein in a first configuration thereof, said inner needle extends along an insertions axis of said probe and in a second configuration thereof, at least a part of said inner needle extends through said opening, creating an angle relative to said insertion axis.

### FIELD

Embodiments of the disclosure relate to a biopsy probe.

### BACKGROUND

Biopsy needles have attained great acceptance within the medical community, since they provide a means for a piecemeal removal of a tissue lesion from human or animal subject for diagnostic or therapeutic propose. Over the years, numerous biopsy needles have been developed according to a specific tissue collection method, such as fine needle aspiration biopsy (FNAB), core needle biopsy (CNB), stereotactic core needle biopsy, vacuum assisted core biopsy.

It is often desirable to sample or test a portion of a tissue from human or animal subjects, particularly in the diagnosis and treatment of potentially cancerous tumors, pre-malignant conditions, and other diseases or disorders. Typically, in the case of tumors, when the physician suspects that cancer or an otherwise diseased condition exists, a biopsy is performed to determine if in fact cells from the tumor are cancerous or otherwise diseased. Many biopsies, such as percutaneous biopsies, are performed with a needle-like instrument used to collect the cells for analysis.

A biopsy needle is usually an elongated hollow body, rigid or flexible having a sharp distal end and a lumen designated to cut and collect tissue samples from a suspected lesion. Typically, biopsy will be taken from an observed lesion in order to retrieve as much as possible relevant matter. Nowadays, needle biopsies are designated to collect tissues of regions on the insertion axis or very close to it but not from locations which are substantially off axis.

In some cases, the off axis locations is significantly relevant to the patients treatment and therefore there is a reason to collect tissues from specific sites on what appears to be the boundary, or margins, of the observed lesion. Currently, there is a growing interest to perform the undesired tissue removal in a minimally invasive way and so to reduce the number of punctures for the biopsy procedure.

### SUMMARY

The following embodiments and aspects thereof are described and illustrated in conjunction with systems, tools and methods which are meant to be exemplary and illustrative, not limiting in scope.

According to some embodiment, there is provided herein a biopsy probe, including: an outer elongated hollow body having a distal end configured to penetrate a sampling region, a proximal end and an opening located between the distal end and the proximal end; an inner needle for obtaining one or more tissue samples, wherein at least a part thereof is located is an inner part of the outer elongated hollow body, wherein in a first configuration thereof, the inner needle extends along an insertions axis of the probe and in a second configuration thereof, at least a part of the inner needle extends through the opening, creating an angle relative to the insertion axis; and a deployable anchoring element located at the distal end of the outer elongated hollow body, the anchoring element is configured to stabilize the biopsy probe when the inner needle is extended through the opening.

According to some embodiment, there is provided herein a biopsy probe, including: an elongated body having a distal end configured to penetrate to a sampling region; a tissue collection element movable relative to the elongated body and configured to collect tissue from a near vicinity of elongated body; and an deployable anchoring mechanism configured to push the tissue collection element in direction perpendicular to a main axis of the elongated body and against soft tissue in an opposite direction. According to some embodiment, the body is an outer elongated hollow body and wherein the tissue collection element is an inner needle, wherein at least a part of the inner needle is located is an inner part of the outer elongated hollow body, wherein in a first configuration thereof, the inner needle extends along an insertions axis of the probe and in a second configuration thereof, at least a part of the inner needle extends through an opening in the outer elongated hollow body, thereby creating an angle relative to the insertion axis.

According to some embodiment, there is provided herein the angle is between 5-120 degrees. The angle may also be between 0 and 180 degrees, for example, between 5-20 degrees, between 10-30 degrees, between 10-50 degrees, between 20-70 degrees, between 10-90 degrees, between 30-120 degrees etc.

According to some embodiment, the inner needle comprises an opening at a distal end thereof, wherein the opening is configured to receive the one or more tissue samples.

According to some embodiment, the probe may further include a vacuum mechanism configured to facilitate insertion of the one or more tissue samples to the opening. The vacuum mechanism may include a vacuum tube. The vacuum tube may include a plurality of holes located at the opening of the inner needle, wherein the plurality of holes are configured to facilitate the insertion of the one or more tissue samples to the opening. The vacuum tube may be movable inside the inner needle.

According to some embodiment, the probe may further include a cutting tool configured to cut the one or more tissue samples located in the opening. The cutting tool may be a tube movable between an inner part of the inner needle and the vacuum tube.

According to some embodiment, the probe may further include a control unit. The control unit may be configured to facilitate manual or automatic control of the inner needle. The control unit may be configured to facilitate manual or automatic control cutting and/or collection of the one or more tissue samples. The control unit may be configured to facilitate manual or automatic control linear and/or rotational movement of the probe. The control unit may be configured to calculate coordinates of sample sites of the tissue samples based on a distance from the insertion axis and the angle relative to the insertion axis.

According to some embodiment, the deployable anchoring element may include an inflatable balloon, a deployable net or a combination thereof.

According to some embodiment, the probe may further include a tissue storage magazine configure to store the sampled tissue, while maintaining relative position of each of the tissue samples.

According to some embodiment, the probe may further include a memory chip to contain coordinates of sample sites of the tissue samples.

According to some embodiment, the inner needle is flexible. According to some embodiment, the inner needle comprises a shape memory material. According to some embodiment, the shape memory material comprises Nitinol. According to some embodiment, the inner needle comprises two or more separate links. According to some embodiment, the two or more separate links are connected to a wire set and controlled by a control unit and/or by a user.

According to some embodiment, there is provided herein a biopsy probe kit, including: a biopsy probe including: an outer elongated hollow body having a distal end configured to penetrate a sampling region, a proximal end and an opening located between the distal end and the proximal end; an inner needle for obtaining one or more tissue samples, wherein at least a part thereof is located is an inner part of the outer elongated hollow body, wherein in a first configuration thereof, the inner needle extends along an insertions axis of the probe and in a second configuration thereof, at least a part of the inner needle extends through the opening, creating an angle relative to the insertion axis; a deployable anchoring element located at the distal end of the outer elongated hollow body, the anchoring element is configured to stabilize the biopsy probe when the inner needle is extended through the opening; and a tissue storage magazine configure to store the tissue samples, while maintaining relative position of each of the tissue samples.

According to some embodiment, there is provided herein a method of performing a biopsy, the method including: inserting an outer elongated hollow body of a biopsy probe into a sampling region; deploying an anchoring element located at a distal end of the outer elongated hollow body; and advancing an inner needle an inner part of the outer elongated hollow body, such that the inner needle extends through an opening in the outer elongated hollow body creating an angle relative to the insertion axis.

According to some embodiment, there is provided herein a biopsy probe, including: an elongated body (such as, but not limited to a tube/hollow body) having a distal end configured to penetrate a sampling region, a tissue collection element (such as but not limited to, a needle, a needle tube, a wire having tissue sampling component on a distal end thereof or any other appropriate element), movable relative to the distal end of the elongated body, for obtaining one or more tissue samples, and a deployable anchoring element, located, for example, at the distal end of the elongated body, the anchoring element is configured to stabilize the biopsy probe when the tissue collection element is obtaining the sample. The tissue collection element may be (at least partially) located inside the elongated body, in case the elongated body is hollow, but may also be (at least partially) located outside the elongated body, in proximity thereto or even movable attached to the elongated body.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 schematically shows an isometric view of a biopsy probe inserted into a body tissue, according to some embodiments;

FIG. 2 schematically shows an isometric view of a biopsy probe inserted into a body tissue, according to some embodiments;

FIG. 3 schematically shows a cross sectional view of a biopsy probe and an isometric view of the probe at the bottom of the figure, according to some embodiments;

FIG. 4 schematically shows a cross sectional view of a biopsy probe and an isometric view of the probe at the bottom of the figure, according to some embodiments;

FIG. 5 schematically shows a cross sectional view of a biopsy probe and an isometric view of the probe at the bottom of the figure, according to some embodiments;

FIG. 6 schematically shows a cross sectional view of a biopsy probe and an isometric view of the probe at the bottom of the figure, according to some embodiments;

FIG. 7a schematically shows a cross sectional view of a biopsy probe and an isometric view of the probe at the bottom of the figure, according to some embodiments;

FIG. 7b schematically shows an isometric view of an inner needle tube of a biopsy probe, according to some embodiments;

FIG. 8a schematically shows a biopsy probe with a control mechanism, according to some embodiments;

FIG. 8b schematically shows a biopsy probe system with a control mechanism and an anchoring element, according to some embodiments;

FIG. 9 schematically shows an isometric view of a biopsy probe being inserted to a body tissue and a schematic illustration of samples taken, according to some embodiments;

FIG. 10 schematically shows a tissue storage magazine for collection of samples taken according to FIG. 9, according to some embodiments; and

FIG. 11 schematically shows an isometric view of a biopsy probe system and the tissue storage magazine according to some embodiments;

FIGS. 12-14a-b schematically show examples of anchoring mechanisms for biopsy probes, according to some embodiments; and

FIG. 15 s schematically shows a side view (top figure) and an isometric view (bottom figure) of an inner needle tube, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

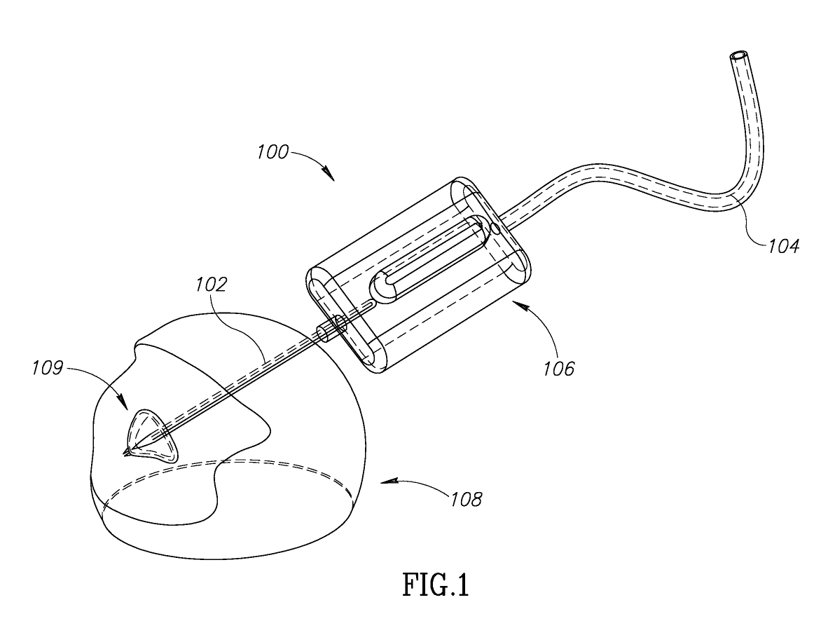

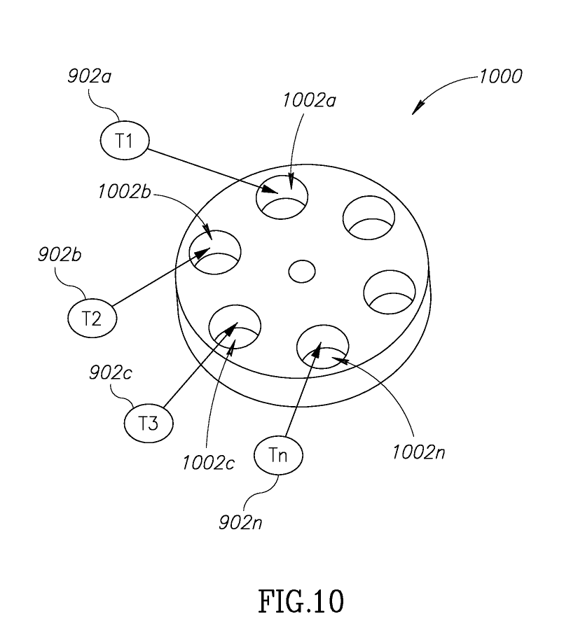

Reference is now made to FIG. 1, which schematically shows an isometric view of a biopsy probe system 100 inserted into a body tissue, according to some embodiments. Biopsy probe system 100 includes a biopsy probe 102, which is configured to be inserted into a body tissue, for example, a body organ 108 in order to retrieve tissue samples from a suspected tissue from a sampling region 109. Sampling region 109 is typically a three dimensional tissue, such as a tumor or a lesion. Since the path of a common, currently used, biopsy probe is confined to the insertion axis of the probe, the only way to obtain samples from a three dimensional suspected tissue is to manipulate, from the outside, the entire probe against the soft tissue while pulling the probe backwards and then pushing it again forward at different angles relative to the soft tissue organ and repeating this multiple times. Such a procedure is unpleasant to the patient, it is not accurate in terms of reaching an exact location and it decreases the ability to maintain the coordinates of the original location of the tissue samples. There are provided herein, according to some embodiments, probe elements (such as biopsy probe 102) that allow retrieving tissue samples along the probe insertion path and at an relative to the insertion path (off the insertion axis). The angle may be between 0 and 180 degrees, for example, between 5-20 degrees, between 10-30 degrees, between 10-50 degrees, between 20-70 degrees, between 10-90 degrees, between 30-120 degrees etc. The distance between the main axis of the needle and the sampling location van be significant (for example, 1-200 mm, 1-100 mm, 5 mm-50 mm, 10-70 mm, etc.). Thus, multiple tissue samples from a three dimensional sampling region, such as sampling region 109, may be obtained by a single biopsy probe insertion. Moreover, the coordinates of the original location of each tissue sample can be maintained (using for example, a sample storage magazine 1000, FIG. 10), wherein tissue samples are collected and recorded according to the sampling order. According to some embodiments, biopsy probe system 100 further includes a control unit 106, configured to control biopsy probe 102 and/or any component thereof, the sampling process, the sampling collection process etc.

According to some embodiments, control unit 106 may contain a set of wires (not shown) in order to control the movement of inner components of biopsy probe 102 as mentioned herein to perform the desirable procedure.

According to some embodiments, control unit 106 may be connected via an electric flexible connection 104 to a mother unit (not shown) to provide power, control analysis, and the like. Each possibility represents a separate embodiment of the invention.

According to some embodiments, control unit 106 may be connected via a wireless connection (not shown) to a mother unit (not shown).

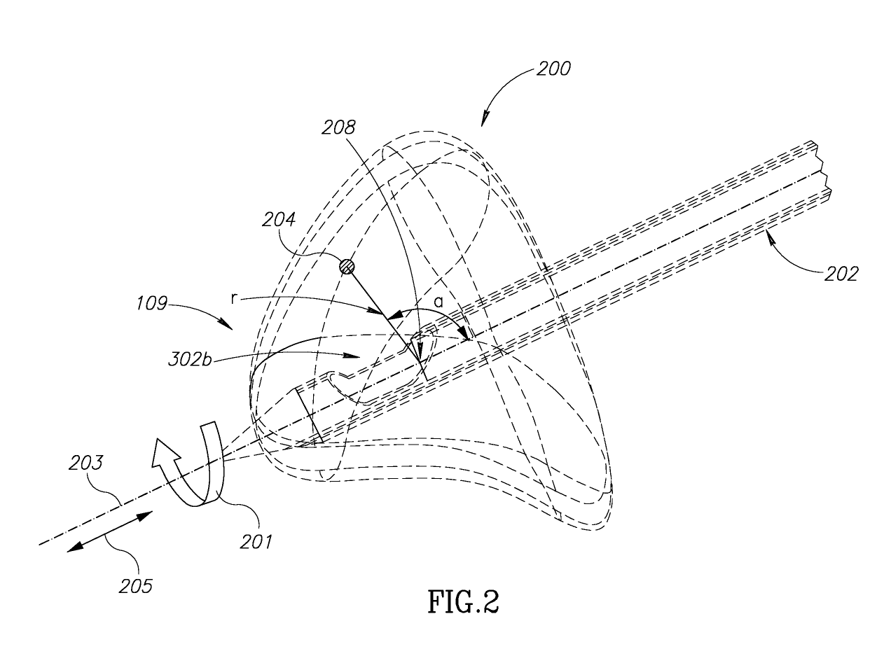

Reference is now made to FIG. 2, which schematically shows an isometric view of a biopsy probe system 200 inserted into a body tissue, according to some embodiments. Biopsy probe 202 may be configured to move in a linear motion 205 and/or in a rotational motion 201 relative to an insertion axis 203, thereby collecting tissue samples along insertion axis 203. However, according to some embodiments, biopsy probe 202 may also be configured to move off insertion axis 203, and thus to obtain tissue samples from a location within sampling region 109 which is off insertion axis 203, such as from a specific tissue sampling location 204. The tissue sampling location 204 from which tissue collection was preformed may be defined by a polar coordinates “r” which is defined as the distance from the proximal end 208 of outer needle opening 302b and the angle “a” which is defined as the angle between “r” and insertion axis 203.

Collection of tissue from off axis locations relative to main insertion axis 203 (in addition to retrieving samples from locations along insertion axis 203) may be achieved while the original location of the samples is calculated and recorded.

According to some embodiments, there is provided herein a biopsy procedure for collection of tissue from off axis locations relative to main insertion axis 203, in addition to collection of tissue samples from locations along insertion axis 203. Such procedure may be controlled by a control unit, such as control unit 106 shown in FIG. 1.

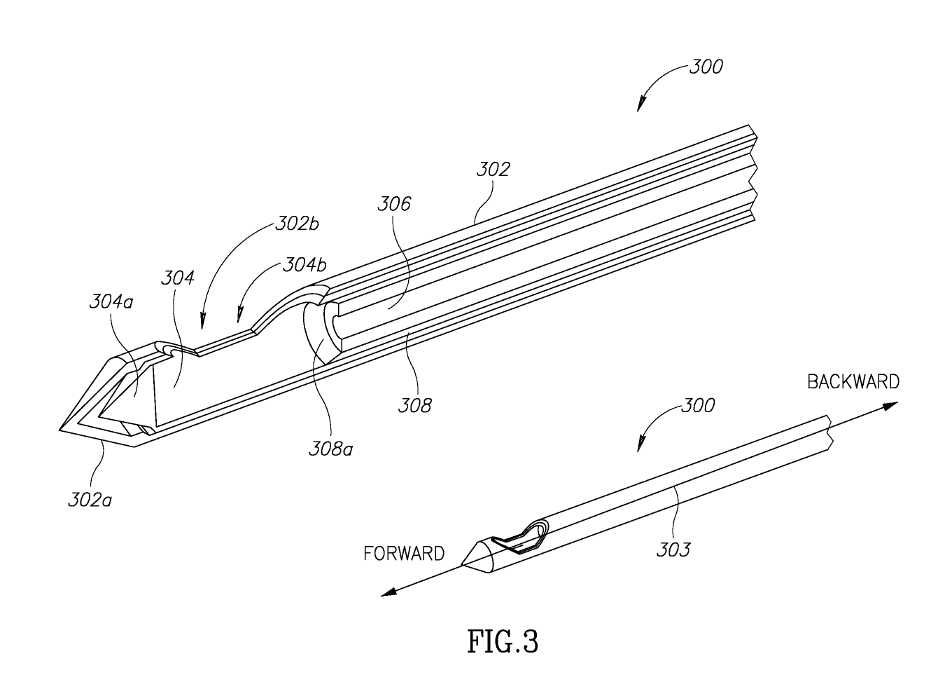

Reference is now made to FIGS. 3-7a, which schematically show cross sectional views of a biopsy probes allowing collection of tissue samples from off axis locations relative to main insertion axis, according to some embodiments. Biopsy probe 300 includes an outer needle tube 302, and an inner needle tube 304, such that at least a portion of inner needle tube 304 is located inside outer needle tube 302. Biopsy probe 300 further includes a cutting tube 308, at least a part thereof is located inside inner needle tube 304 and a vacuum tube 306, at least a part thereof is located inside inner needle tube 304. The term “outer needle tube” and “outer elongated hollow body” may be interchangeably used. The term “inner needle tube” and “inner needle” may be interchangeably used.

Each one of outer needle tube 302 and inner needle tube 304 have sharp distal end sections: outer needle sharp distal end 302a and inner needle sharp distal end 304a, respectively, which allow smooth tissue penetration.

Each one of outer needle tube 302 and inner needle tube 304 have openings at distal ends thereof: outer needle tube opening 302b and inner needle opening 304b, respectively. These opening are configured for tissue collection, while outer needle tube opening 302b also enables inner components of the probe to be extended at an angle to off axis locations relative to main insertion axis 303 for tissue collection. Inner components may include inner needle tube 304, cutting tube 308, cutting tool 308a, and vacuum tube 306 and optionally other components. Each possibility represents a separate embodiment of the invention.

As shown in FIG. 3 inner needle tube 304 is extended along main insertion axis 303 all the way through outer needle tube 302, such that inner needle opening 304b essentially overlaps with outer needle tube opening 302b and outer needle sharp distal end 302a essentially overlaps with inner needle sharp distal end 304a. This configuration allows collection of tissue samples along main insertion axis 303. In FIG. 3 cutting tool 308a is shown to be retrieved backwards.

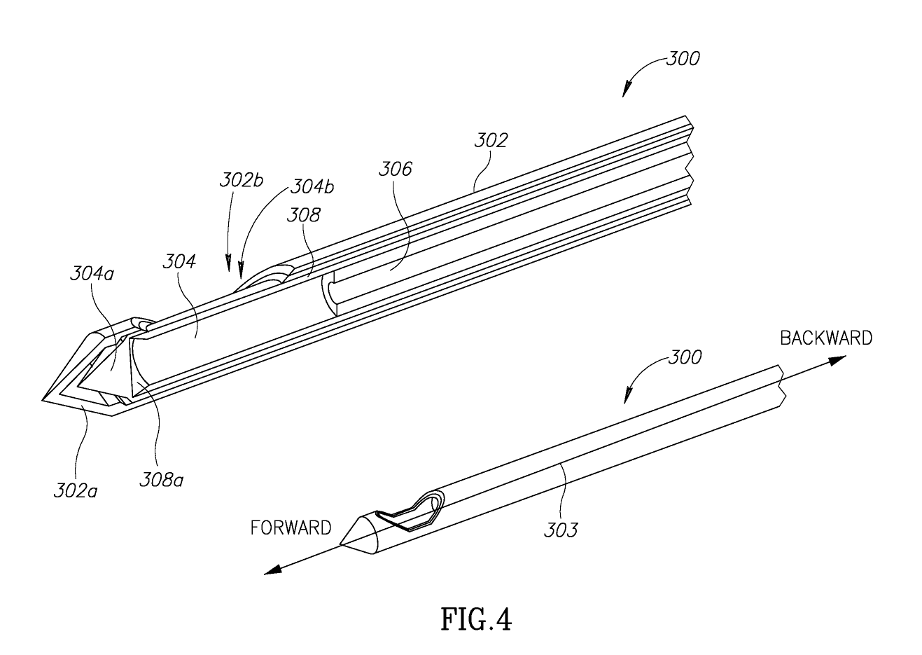

As shown in FIG. 4 cutting tool 308a is extended distally such that upon this extension, cutting tool 308a is designed to cut tissue collected in inner needle opening 304b and thus to separate the sampled tissue from the bulk tissue. This configuration also allows penetration of biopsy probe 300 into the sampling region.

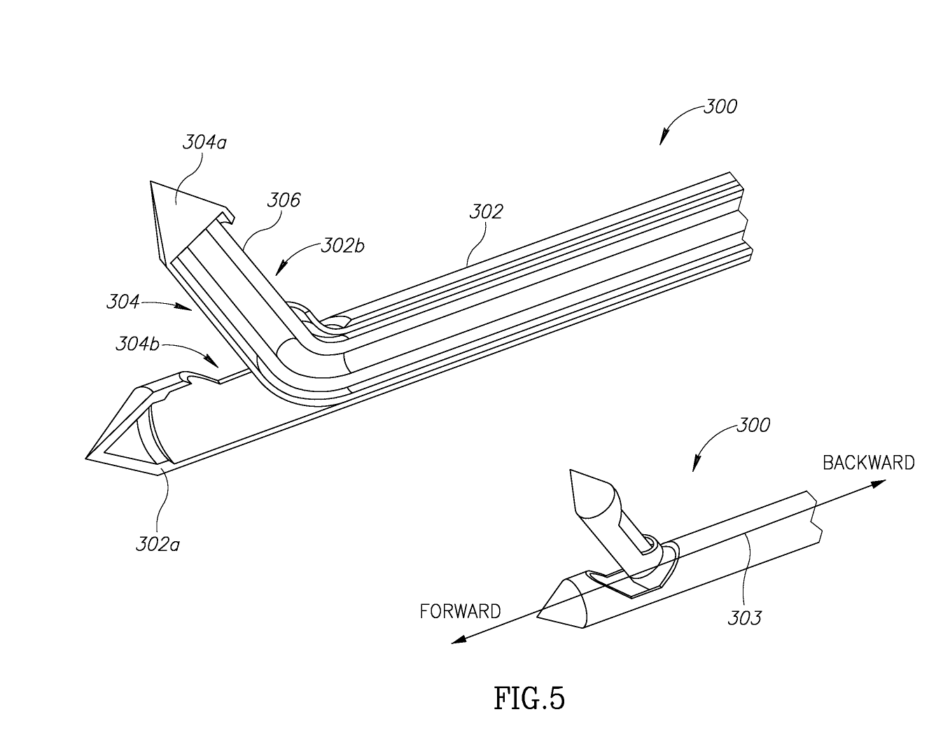

As shown in FIG. 5 inner needle tube 304 was pulled proximally (backwards) such that the distal end thereof could be released, exit through outer needle tube opening 302b and bent away from outer needle tube 302 creating an angle between the distal portion of inner needle tube 304 and outer needle tube 302.

According to some embodiments, to achieve such configuration, inner needle tube 304 may be made of a shape memory material (such as a shape memory alloy, for example, Nitinol, Fe—Mn—Si, Cu—Zn—Al, Cu—Al—Ni or any other suitable material). Thus, when inner needle tube 304 is released through outer needle tube opening 302b, it returns to its original predetermined shape, which determines its path through the tissue and to the desired sampling location by cutting through the tissue using inner needle sharp distal end 304a. While inner needle tube 304 passes (cuts) through the tissue towards the desired sampling location, vacuum tube 306 blocks inner needle opening 304b. According to some embodiments, in case of a biopsy procedure that requires to sample from areas located at different angles the inner needle tube 304 may need to be replaced each time to a shape-memory alloy material with a different pre-deformed shape.

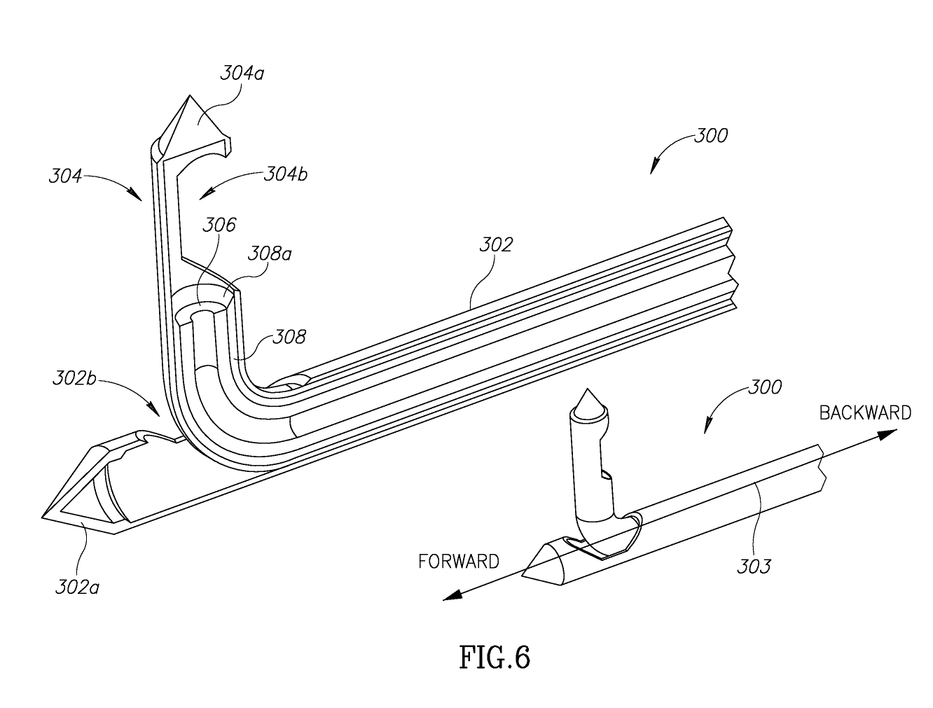

As shown in FIG. 6 once inner needle tube 304 has reaches its final desired sampling location, vacuum tube 306 is pulled backwards to expose inner needle opening 304b. Vacuum is applied, such that tissue is sucked into and collected in inner needle opening 304b, while outer needle sharp distal end 302a serves as an anchor in the collection process and stabilizes biopsy probe 300.

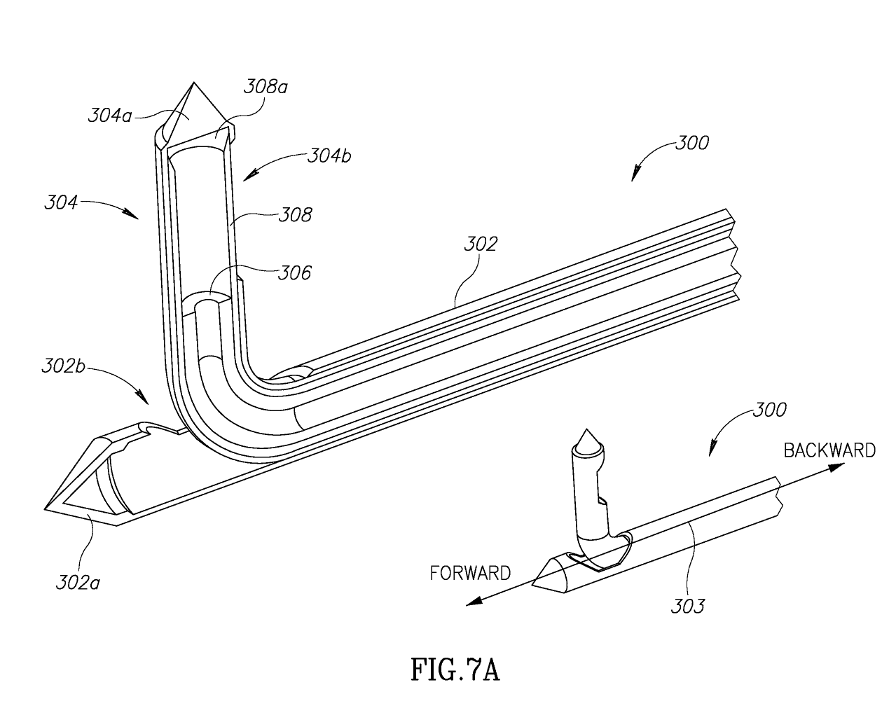

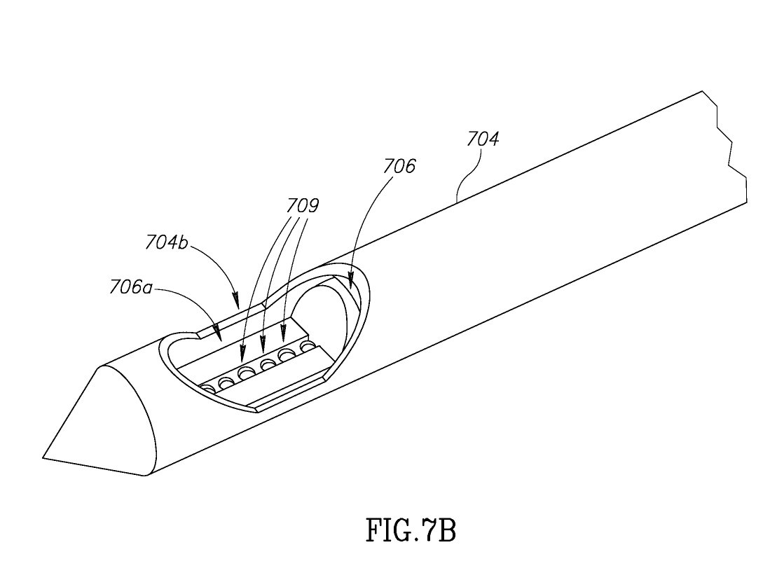

As shown in FIG. 7a, cutting tube 308 moves towards inner needle distal end 304a to cut, using cutting tool 308a, the tissue sampled in inner needle opening 304b. Vacuum tube 306 is pulled backwards while sucking the tissue sample. Outer needle sharp distal end 302a may still serve as an anchor.

According to some embodiments, vacuum tube 306 may be made from a flexible or partially flexible material in order to be able to curve with inner needle tube 304 as it moves to off axis locations relative to the main insertion axis 303.

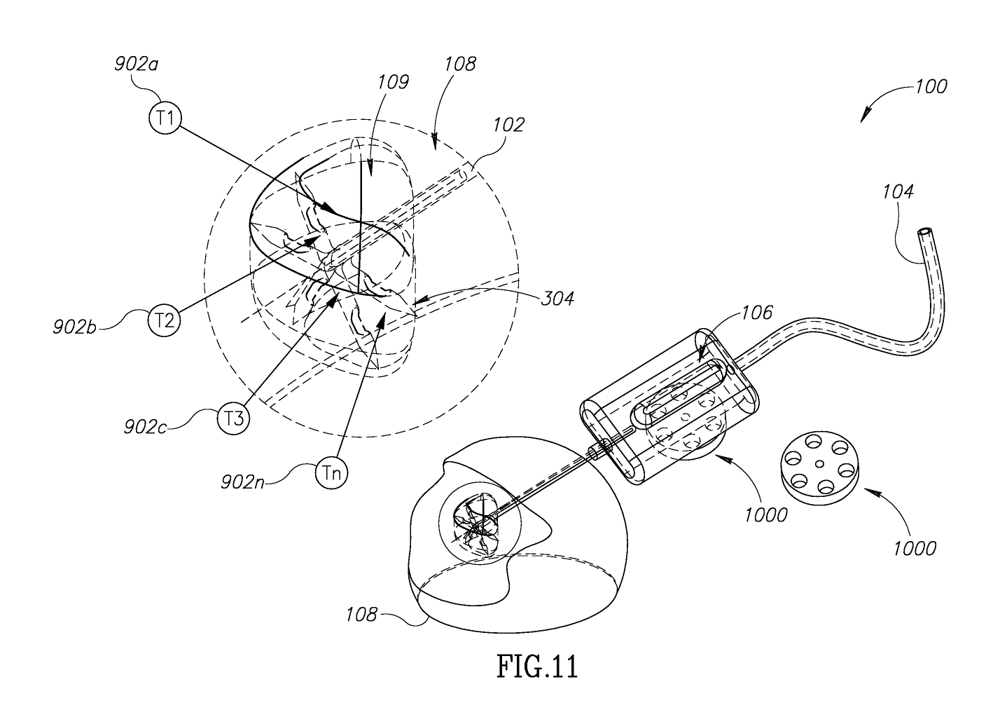

Reference is now made to FIG. 7b, schematically shows an isometric view of an inner needle tube of a biopsy probe, according to some embodiments. According to some embodiments, the inner needle tube 704 may include therein a vacuum tube 706 shaped such that at least a distal section 706a thereof is flat and includes multiple (for example two or more) suction holes 709, which are configured to facilitate suction of the sampled tissue into a larger volume of inner needle opening 704b. This form may prevent or minimize blocking of the vacuum tube by the sampled tissue as it moves into inner needle opening 704b. According to some embodiments, this form may reduce or prevent early vacuum blocking by the tissue as it moves inside inner needle opening 704b or outer needle opening (not shown). Furthermore, this configuration of vacuum tube 706 facilitates the attachment of the sampled tissue to vacuum tube distal end (which, functionally, according to some embodiments, serves as a “spoon” carrying the tissue sample), and the extraction of the tissue sample in its original form from the collection point to the tissue collection magazine (for example as shown in FIGS. 11 and 11). The Extraction of the tissue sample is performed, while the vacuum tube 706 is pulled back (proximally) pulling the tissue sample that is attached to its distal section 706a. According to some embodiments, a control unit such as a control unit 106 may control biopsy probe 300 and/or any components thereof, the sampling process, the sampling collection process etc.

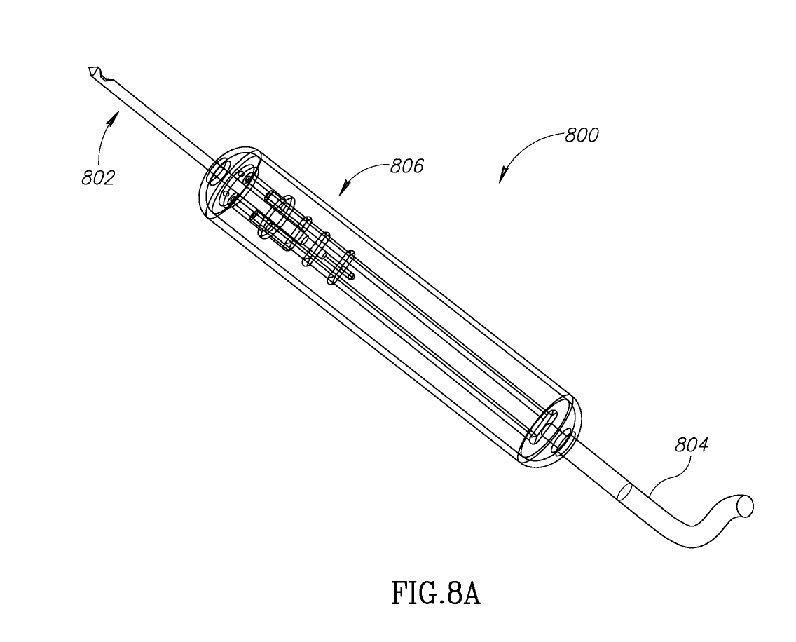

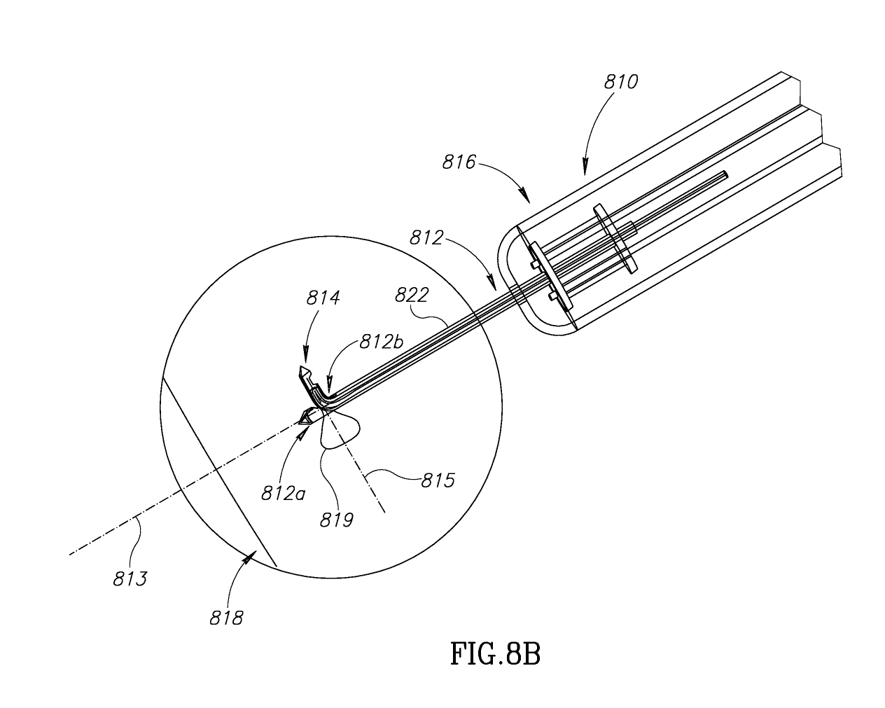

Reference is now made to FIG. 8a, which schematically shows a biopsy probe system 800 having a biopsy probe 802, a control mechanism 806, and an electric connection 804 (similar to electric connection 104), according to some embodiments.

Reference is now made to FIG. 8b, which schematically shows a biopsy probe system 810 including a biopsy probe 812, control mechanism 816 and an anchoring element 819, according to some embodiments.

Enhanced anchoring of biopsy probe 812 (having outer needle tube 822 and inner needle tube 814, shown herein in a position perpendicular (along axis 815) to outer needle tube 822) in the sampling region may be achieved by deploying anchoring element 819, which is attached to or integrally formed with outer needle distal end 812a of biopsy probe 812, in location opposite to outer needle opening 812b. Anchoring element 819 may include for example, a deployable net or an inflatable balloon, which are configured to deploy/inflate after insertion of biopsy probe 812 to the sampling region 818 and before releasing inner needle tube 814 to an off axis locations relative to the main insertion axis 813. Anchoring element 819 has a spatial (three dimensional) shape when deployed and is thus configured to increase the stability of biopsy probe 812 in all directions (in addition to the direction of the main insertion axis 813) during the sampling process.

According to some embodiments, an example of a mode of operation of the biopsy probe (such as biopsy probe 300) is described hereinbelow:
1. The biopsy probe (such as biopsy probes 102, 300, 802, 812) is inserted to the sampling region (such as sampling region 109);
2. The anchoring element (such as anchoring element 819, 1202, 1302) is deployed;
3. The inner needle tube (such as inner needle tube 304, 704, 814) is pulled backwards inside the outer needle tube (such as outer needle tube 302, 822) so that inner needle distal end (such as inner needle distal end 304a) reaches the outer needle opening (such as outer needle opening 302b);
4. The inner needle tube (such as inner needle tube 304, 704, 814) is moved forward such that the inner needle distal end (such as inner needle distal end 304a) passes through the outer needle opening (such outer needle opening 302b) to a desired off axis location relative to the insertion axis (such as insertion axis 203, 303, 813, 1513) in order to collect a tissue sample;
5. The vacuum tube (such as vacuum tube 306, 706) is pulled backwards to expose the inner needle opening 304b (such as inner needle opening 304b);
6. Vacuum is applied to the tissue via the vacuum tube (such as vacuum tube 306, 706) so that tissue samples will be pulled (sucked) the inner needle opening (such as into inner needle opening 304b, 704b, 1502b);
7. The cutting tube (such as cutting tube 308) is moved forwards towards the inner needle distal end (such as inner needle distal end 304a) to cut and collect the tissue sample; and
8. The vacuum tube (such as vacuum tube 306, 706) is pulled back with the tissue sample.

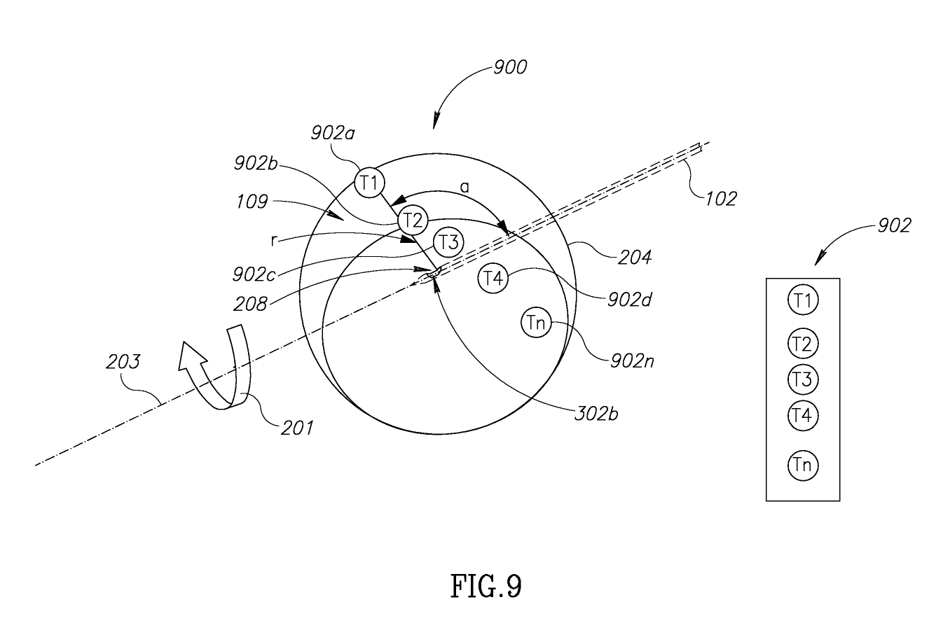

Reference is now made to FIG. 9, which schematically shows an isometric view of a biopsy probe system 900 being inserted to a body tissue and a schematic illustration of samples taken, according to some embodiments. Tissue may be collected from all locations inside sampling region 109 using biopsy probe 102. The tissue sampling location 204 from which tissue collection was preformed may be defined by a polar coordinates “r” which is defined as the distance from the proximal end 208 of outer needle opening 302b and the angle “a” which is defined as the angle between “r” and insertion axis 203.

Each tissue sample 902 collected by biopsy probe system 900 is marked as t1 902a, t2 902b, t3 902c, t4 902d . . . tn 902n and is tagged with its own “coordinates” “r” “a”.

Biopsy probe 102 is shown having multiple configurations as described herein, which result from linear and/or rotational (201) motion of biopsy probe 102 relative to an insertion axis and from the movement of inner needle tube such as inner needle tube 304 (FIGS. 3-7) in order to obtain tissue samples from various locations of the three dimensional sampling region 109.

Reference is now made to FIG. 10, which schematically shows a magazine for collection of samples taken according to FIG. 9, according to some embodiments. The tissue samples t1 902a, t2 902b, t3 902c . . . tn 902n are delivered from their location to a tissue storage magazine 1000, which has a dedicated chamber for each tissue sample so that each storage chamber c1 1002a, c2 1002b, c3 1002c . . . cn 1002n in the magazine contains only one tissue sample from its unique location. Tissue storage magazine 1000 is built in a way that allows the sampled tissue removal from the device and to be replaced and used by a pathologist as part as the histology slide preparation.

According to some embodiments, tissue storage magazine 1000 may be filled with formalin in order to allow for pathology block preparation. According to some embodiments, tissue storage magazine 1000 may contain a memory chip (not shown), which records the coordinate of the stored tissue in each storage chamber. The memory chip contains the exact coordinates of the site from which the tissue was collected with the corresponding chamber identification.

Reference is now made to FIG. 11, which schematically shows an isometric view of a biopsy probe system and the tissue storage magazine according to some embodiments. Tissue storage magazine 1000 is a removable module designated to locate sampled tissues from various locations in a body organ in an ordered way. Tissue samples 902 (a, b, c, . . . n) are delivered to tissue storage magazine 1000, using a vacuum tube (for example, FIG. 3, 306). Tissue storage magazine 1000 has a dedicated chamber for each tissue sample such that each storage chamber c1 FIG. 10, 1002a, c2 FIG. 10, 1002b, . . . cn (FIG. 10, 1002n) in the magazine, contains only one respective tissue sample t1 902a, t2 902b . . . tn 902n from its unique location in an ordered manner. For example, first tissue sample t1 902a is delivered to location c1 FIG. 10, 1002a, sample t2 902b is delivered to location c2 FIG. 10, 1002b, sample to 902n is delivered to location cn, 1002n (FIG. 10). The tissue storage magazine 1000 may be attached to control unit 106 in order to allow storage of the tissue samples taken from sampling region 109 of organ 108. Biopsy probe 102 is shown having multiple configurations as described herein, which result from linear and/or rotational motion of biopsy probe 102 relative to an insertion axis and from the movement of inner needle tube such as inner needle tube 304 (FIGS. 3-7) in order to obtain tissue samples from various locations of the three dimensional sampling region 109.

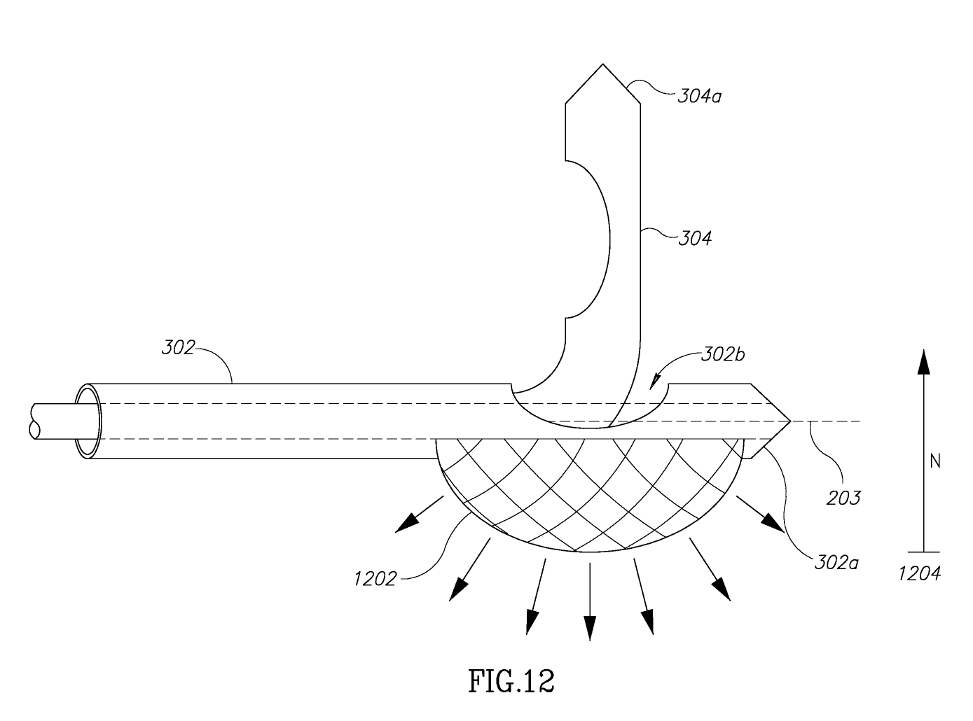

Reference is now made to FIGS. 12-14a-b, which schematically show examples of anchoring mechanisms for biopsy probes, according to some embodiments. As shown in FIG. 12, enhanced anchoring may be achieved by an anchoring element, such as an inflatable balloon, 1202 to outer needle tube 302 in a location opposite to outer needle opening 302b. Anchoring element 1202, which may be deployable (such as an inflatable balloon) and configured to be deployed/inflated after insertion of the biopsy probe to the sampling region, increases the effective surface of outer needle tube 302 such that it can resists higher forces (in a direction perpendicular to insertion axis 203 but also in other directions) than the resistance of the tissue to outer needle sharp distal end 302a due to an enhanced Normal force exerted by the tissue surrounding anchoring element 1202. The enhanced net force is directed at a direction 1204, which supports the movement and penetration of inner needle tube distal end 304a while inner needle tube 304 propagates via outer needle opening 302b to an off axis locations relative to insertion axis 203.

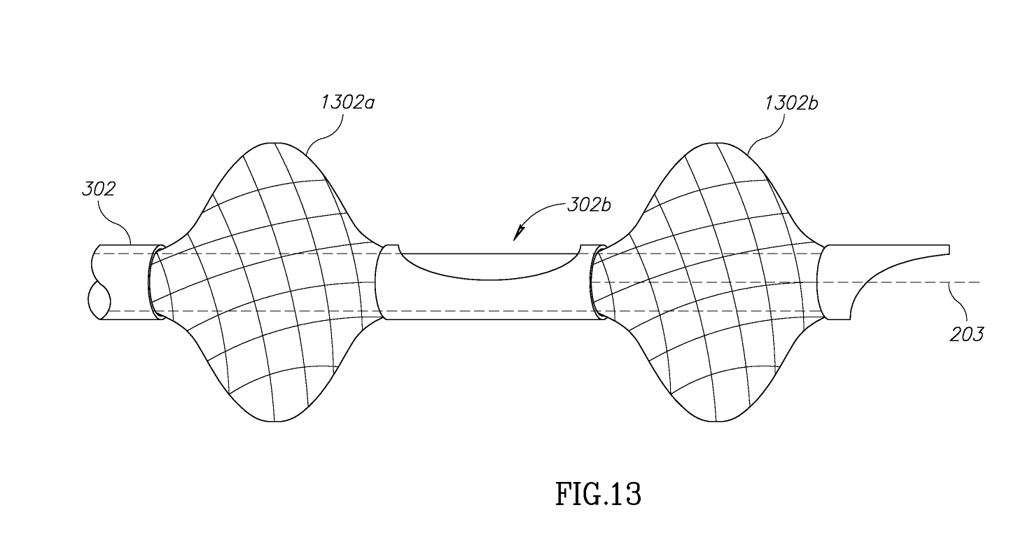

As shown in FIG. 13, enhanced anchoring may be achieved by anchoring element 1302a and b connected to outer needle tube 302 located on both sides of outer needle opening 302b. Anchoring element 1302a is located proximally to outer needle opening 302b and anchoring element 1302b is located distally to outer needle opening 302b. Anchoring elements 1302a and 1302b may be circumferentially located around outer needle tube 302. Anchoring elements 1302a and 1302b may be deployed only after outer needle tube 302 is inserted into the tissue. The enhanced net force is directed at a direction 1204, which supports the movement and penetration of inner needle tube distal end, while inner needle tube (not shown) propagates via outer needle opening 302b to an off axis locations relative to insertion axis 203.

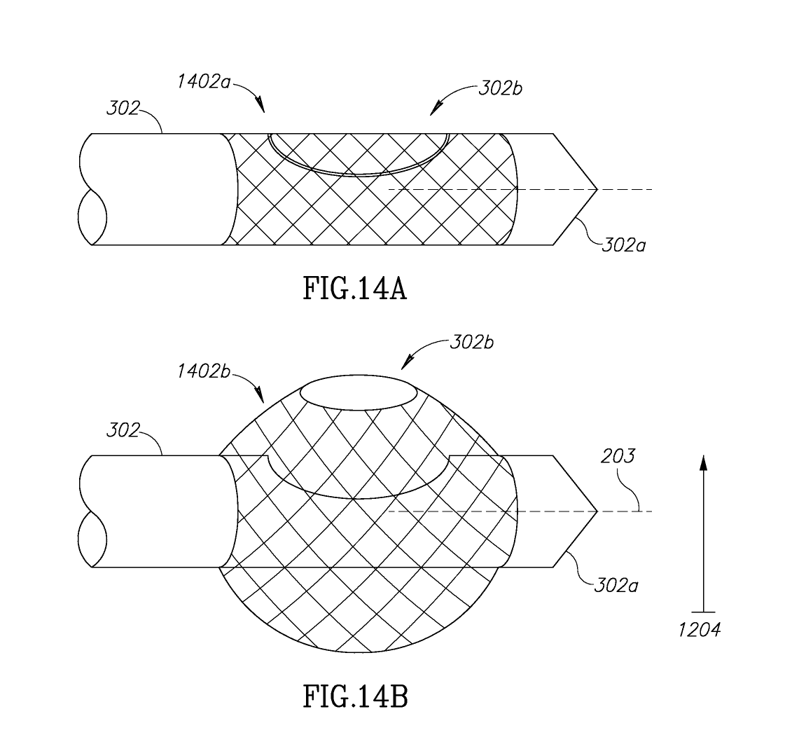

FIG. 14a and FIG. 14b show another option of an anchoring net 1402 in a collapsed configuration (1402a) and in a deployed configuration (1402b), respectively. After outer needle tube 302 is inserted to a target location using outer needle sharp distal end 302a, enhanced anchoring may be achieved deploying anchoring net 1402. Anchoring net 1402 should be designed not to block outer needle opening 302b in order to allow the sampling process from off axis location relative to insertion axis 203.

When deployed (1402b), anchoring net 1402 is designed to increase the effective surface of the outer needle tube 302 such that it can resist higher forces (in a direction perpendicular to insertion axis 203) than the resistance of the tissue to outer needle sharp distal end (not shown), due to an enhanced Normal force exerted by the tissue encompassing expanded outer needle tube 302. The enhanced net force is directed at a direction 1204 which supports the movement and penetration of inner needle tube distal end (not shown) while inner needle tube (not shown) propagates via outer needle opening 302b to off axis locations relative to insertion axis 203.

Other anchoring elements/mechanisms may be used.

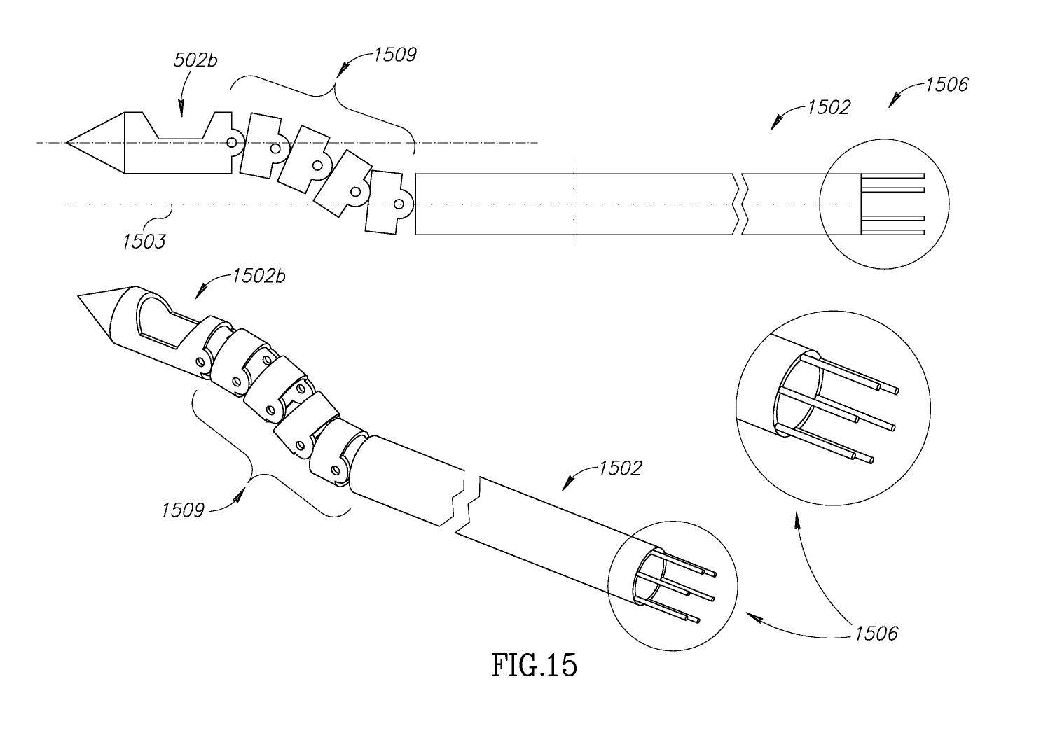

As disclosed herein, according to some embodiments of this disclosure, inner needle tube may be made of a shape memory material allowing it to obtain its original form after its distal end exits through an opening in the outer needle tube. This allows penetration of the inner needle to sampling areas remote from the probe axis. However, other forms, configurations and/or materials may be used, according to some embodiments, to enable the inner needle to penetrate through soft tissue and to reach sampling locations, which are remote from the probe axis. An example of such configuration is presented in FIG. 15, which schematically shows a side view (top figure) and an isometric view (bottom figure) of an inner needle tube, according to some embodiments. Inner needle tube 1502 or a portion thereof may include links/vertebras 1509 which allow manipulation of inner needle tube 1502 in the tissue, after it exits through an opening in the outer needle tube (not shown). This allows penetration of inner needle tube 1502 to sampling areas remote from the probe axis and from insertion axis 1503 (off axis). The actual tissue sampling is performed through inner needle opening 1502b. The sampling process may be the same or similar to that described hereinabove. Links/vertebras 1509 are shown in this figure to have four link/vertebra members, but inner needle tube 1502 may include any number of link/vertebra members (such as 2, 3, 4, 5, 5-10 or more). Links/vertebras 1509 may be separated from each other and connected by wire(s) or may be partially connected to each other. Links/vertebras 1509 are also connected a set of control wires 1506 connectable to a control unit (not shown). Control wires 1506 are configured to control the movement of link Links/vertebras 1509 (for example, separately control each link) and thus to facilitate manual or automatic control of inner needle tube 1502 movement inside the tissue. While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

1. A biopsy probe, comprising:
an outer elongated hollow body having a distal end configured to penetrate a sampling region, a proximal end and an opening located between said distal end and said proximal end;
an inner needle for obtaining one or more tissue samples, wherein at least a part thereof is located in an inner part of said outer elongated hollow body, wherein in a first configuration thereof, said inner needle extends along an insertions axis of said probe and in a second configuration thereof, at least a part of said inner needle extends through said opening, creating an angle relative to said insertion axis; and
a deployable anchoring element located at said distal end of said outer elongated hollow body, said anchoring element is configured to stabilize said biopsy probe when said inner needle is extended through said opening.

2. The biopsy probe according to claim 1, wherein said angle is between 5-120 degrees.

3. The biopsy probe according to claim 1, wherein said inner needle comprises an opening at a distal end thereof, wherein said opening is configured to receive said one or more tissue samples.

4. The biopsy probe according to claim 3, further comprising a vacuum mechanism configured to facilitate insertion of said one or more tissue samples to said opening.

5. The biopsy probe according to claim 4, wherein said vacuum mechanism comprises a vacuum tube.

6. The biopsy probe according to claim 5, wherein said vacuum tube is in fluid flow connection with a plurality of holes located at said opening of said inner needle, wherein said plurality of holes are configured to facilitate said insertion of said one or more tissue samples to said opening.

7. The biopsy probe according to claim 5, wherein said vacuum tube is movable inside said inner needle.

8. The biopsy probe according to claim 5, further comprising a cutting tool configured to cut said one or more tissue samples located in said opening.

9. The biopsy probe according to claim 8, wherein said cutting tool is a tube movable between an inner part of said inner needle and said vacuum tube.

10. The biopsy probe according to claim 1, further comprising a control unit configured to facilitate manual or automatic control of said inner needle.

11. The biopsy probe according to claim 1, further comprising a control unit configured to facilitate manual or automatic control cutting and/or collection of said one or more tissue samples.

12. The biopsy probe according to claim 1, further comprising a control unit configured to facilitate manual or automatic control linear and/or rotational movement of said probe.

13. The biopsy probe according to claim 1, wherein said a deployable anchoring element comprises an inflatable balloon, a deployable net or a combination thereof.

14. The biopsy probe according to claim 1, further comprising a tissue storage magazine configure to store the sampled tissue, while maintaining relative position of each of said tissue samples.

15. The biopsy probe according to claim 1, further comprising a control unit configured to calculate coordinates of sample sites of said tissue samples based on a distance from said insertion axis and the angle relative to said insertion axis.

16. (canceled)

17. The biopsy probe according to claim 1, wherein said inner needle is flexible and/or comprises a shape memory material.

18. (canceled)

19. (canceled)

20. The biopsy probe according to claim 1, wherein said inner needle comprises two or more separate links.

21. The biopsy probe according to claim 1, wherein said two or more separate links are connected to a wire set and controlled by a control unit and/or by a user.

22. A biopsy probe kit, comprising:
a biopsy probe comprising:
an outer elongated hollow body having a distal end configured to penetrate a sampling region, a proximal end and an opening located between said distal end and said proximal end; and
an inner needle for obtaining one or more tissue samples, wherein at least a part thereof is located is an inner part of said outer elongated hollow body, wherein in a first configuration thereof, said inner needle extends along an insertions axis of said probe and in a second configuration thereof, at least a part of said inner needle extends through said opening, creating an angle relative to said insertion axis; and
a deployable anchoring element located at said distal end of said outer elongated hollow body, said anchoring element is configured to stabilize said biopsy probe when said inner needle is extended through said opening; and
a tissue storage magazine configure to store said tissue samples, while maintaining relative position of each of said tissue samples.

23. A method of performing a biopsy, the method comprising:
inserting an outer elongated hollow body of a biopsy probe into a sampling region;
deploying an anchoring element located at a distal end of the outer elongated hollow body; and
advancing an inner needle an inner part of the outer elongated hollow body, such that the inner needle extends through an opening in the outer elongated hollow body creating an angle relative to said insertion axis.

24. (canceled)

25. (canceled)
